(19) United States
(12) Patent Application Publication
(10) Pub. No.: US 2019/0290200 A1
(43) Pub. Date: Sep. 26, 2019

(54) PROBE HOLDER FOR BRAIN FUNCTION MEASUREMENT DEVICE AND BRAIN FUNCTION MEASUREMENT DEVICE

(71) Applicant: Shimadzu Corporation, Kyoto (JP)

(72) Inventor: Akihiro Ishikawa, Kyoto (JP)

(73) Assignee: Shimadzu Corporation, Kyoto (JP)

(21) Appl. No.: 15/928,482

(22) Filed: Mar. 22, 2018

(51) Int. Cl. A61B 5/00; A61B 5/0478

(52) U.S. Cl. CPC A61B 5/6803; A61B 5/0478; A61B 5/0059

(57) ABSTRACT

A probe holder for a brain function measurement device is provided with a holder portion including a probe attachment portion for attaching a measuring probe of the brain function measurement device, disposed on a subject's frontal region side, and deformable in accordance with the subject's head shape, a holder fixing portion disposed on the subject's occipital region side and formed in a loop shape such that a middle portion of the occipital region is fitted into the holder fixing portion, and a connecting unit connecting the holder portion on the frontal region side and the holder fixing portion on the occipital region side to each other.

### FIELD

The present invention relates to a probe holder for a brain function measurement device and a brain function measurement device and, more particularly, to a probe holder for a brain function measurement device for disposing a measuring probe on a test subject's head and a brain function measurement device provided with a probe holder.

### BACKGROUND

In the related art, a configuration in which a measuring probe is attached to a holder disposed on a test subject's head during brain function measurement is known (see, for example, Patent Literature 1).

In Patent Literature 1, a probe holder provided with a plurality of probe attachment portions for attaching a measuring probe is disclosed. The probe holder includes a holder portion disposed on a subject's forehead with the plurality of probe attachment portions formed in the holder portion and one belt connecting lateral end portions on both sides of the holder portion to each other through the subject's occipital region side. In other words, the probe holder is formed in a head band shape making a circle through the occipital region side from the subject's forehead and is fixed to a predetermined position in the subject's head by a tightening force being applied to the head by the holder portion and the belt.

[Patent Literature 1] JP-A-2008-200226

### SUMMARY

The head band-shaped probe holder disclosed in Patent Literature 1 has a problem in that the probe holder can be stably fixed, with the head fitting in the probe holder, only in some cases as, for example, subjects' individual head size differences and individual head shape differences result in different mounting parts (parts coming into contact with heads). In this case, it takes time to fix the probe holder during measurement or a varying brain function measurement result arises as the probe holder becomes likely to deviate during the measurement. Desired in this regard is a probe holder that can be fixed to a test subject's head in a more stable manner.

The present invention has been made to solve the above problem, and an object of the present invention is to provide a probe holder that can be fixed to a test subject's head in a more stable manner and a brain function measurement device that is provided with the probe holder.

In order to achieve the above object, a probe holder for a brain function measurement device according to a first aspect of the present invention includes a holder portion including a probe attachment portion for attaching a measuring probe of the brain function measurement device, disposed on a subject's frontal region side, and deformable in accordance with the subject's head shape, a holder fixing portion disposed on the subject's occipital region side and formed in a loop shape such that a middle portion of the occipital region is fitted into the holder fixing portion, and a connecting unit connecting the holder portion on the frontal region side and the holder fixing portion on the occipital region side to each other.

In the probe holder for a brain function measurement device according to the first aspect of the present invention, a loop-shaped contact surface can be formed on the test subject's occipital region side by the holder fixing portion since the holder fixing portion disposed on the subject's occipital region side and formed in a loop shape such that the middle portion of the occipital region is fitted into the holder fixing portion is disposed as described above. As a result, the holder portion on the frontal region side can be stably supported from the occipital region side by the loop-shaped (annular) contact surface into which the middle portion of the occipital region is fitted unlike in a case where a belt-shaped contact surface simply for placing a belt like a bridge is formed in the occipital region. In addition, the holder fixing portion can be mounted with the middle portion of the rounded occipital region fitted inside the loop shape, and thus the head is allowed to fit more in the holder fixing portion than in conventional structures even in the event of individual head shape differences. As a result, the holder portion on the frontal side and the holder fixing portion on the occipital side are allowed to be in closer contact with the head of the subject, and thus the probe holder can be fixed to the head of the test subject in a more stable manner.

Preferably, in the probe holder for a brain function measurement device according to the first aspect described above, the holder fixing portion is formed such that an upper portion of the loop shape is disposed at the same height as an upper end portion of the holder portion or closer to a centriciput side than the upper end portion of the holder portion and a lower portion of the loop shape is disposed on a lower portion side of the occipital region on the subject's head. With this configuration, the holder fixing portion can be disposed in a wider range of the head of the test subject in an up-down direction, and thus the holder fixing portion can be fixed to the head of the test subject in a more stable manner. As a result, deviation of the probe holder from the head during the mounting can be effectively suppressed.

Preferably, in the probe holder for a brain function measurement device according to the first aspect described above, the holder fixing portion is formed in the loop shape surrounding the subject's occipital region. With this configuration, the holder fixing portion can be mounted with the entire occipital region fitted inside the loop shape, and thus the stability of the holder fixing portion during the mounting can be further improved.

Preferably, in the probe holder for a brain function measurement device according to the first aspect described above, the holder fixing portion includes a fixing member connected to the holder portion via the connecting unit and a first adjustment portion constituting a part of the loop shape by being connected to the fixing member and configured to be adjustable in length. With this configuration, the length of the loop-shaped part of the holder fixing portion can be adjusted in accordance with the subject's head shape, and thus the probe holder can be fixed to the head of the test subject in a more stable manner even in the event of individual head shape differences.

Preferably, in the probe holder for a brain function measurement device according to the first aspect described above, the connecting unit is disposed on each of the subject's left and right temporal region sides and a pair of the connecting units is disposed on each temporal region side such that each of the upper end portion and a lower end portion of the holder portion is connected to the holder fixing portion. With this configuration, each of the upper end portion and the lower end portion of the holder portion can be supported by the holder fixing portion on the occipital side via the connecting unit. As a result, the subject's frontal region is allowed to fit more in the holder portion when the holder portion is fixed, and thus the occurrence of positional deviation of the holder portion (measuring probe) during the mounting of the probe holder can be effectively suppressed.

Preferably, in the probe holder for a brain function measurement device according to the first aspect described above, the holder fixing portion includes an attachment portion extending from the subject's occipital region side toward the temporal region side and the connecting unit connects the attachment portion and the holder portion to each other. With this configuration, the attachment portion extending from the occipital side to the temporal region side can be connected to the holder portion on the frontal side, and thus the length of the connection between the holder portion and the holder fixing portion by the connecting unit can be decreased. A tensile force (that is, a force to tighten the head of the test subject) acts on the connecting unit so that the holder portion and the holder fixing portion are fixed to the head, and thus a large tensile force is allowed to act with ease as a result of the decrease in connection length (length of the connecting unit). As a result, the holder portion and the holder fixing portion can be fixed in close contact to the head in a more appropriate manner.

Preferably, in the probe holder for a brain function measurement device according to the first aspect described above, the connecting unit is a belt-shaped member in which one end is fixed to one of the holder fixing portion and the holder portion and the other end side is connected to the other one of the holder fixing portion and the holder portion, a second adjustment portion disposed in the other one of the holder fixing portion and the holder portion for adjusting a connection length of the connecting unit between the holder fixing portion and the holder portion is further provided, and the second adjustment portion includes a passage portion through which the other end of the connecting unit passes and a switching operation unit switching between a state where the connecting unit is fixed in the passage portion and a state where the connecting unit is movable in the passage portion. With this configuration, during the mounting of the probe holder, the connection length of the connecting unit can be easily and conveniently adjusted simply by the switching operation unit being operated. As a result, the probe holder can be fixed to the head in a more stable manner, by the holder portion and the holder fixing portion connected at an appropriate connection length, even in the case of subjects' individual head shape differences.

A brain function measurement device according to a second aspect of the present invention includes a light output unit irradiating a subject's head with measuring light via a measuring probe, a light detection unit receiving the measuring light passing through the subject's head via the measuring probe, and a probe holder for fixing the measuring probe to the subject's head, in which the probe holder includes a holder portion including a probe attachment portion for attaching the measuring probe, disposed on the subject's frontal region side, and deformable in accordance with the subject's head shape, a holder fixing portion disposed on the subject's occipital region side and formed in a loop shape such that a middle portion of the occipital region is fitted into the holder fixing portion, and a connecting unit connecting the holder portion on the frontal region side and the holder fixing portion on the occipital region side to each other.

In the brain function measurement device according to the second aspect of the present invention, a loop-shaped contact surface can be formed on the test subject's occipital region side by the holder fixing portion of the probe holder since the holder fixing portion disposed on the subject's occipital region side and formed in a loop shape such that the middle portion of the occipital region is fitted into the holder fixing portion is disposed in the probe holder as described above. As a result, the holder portion on the frontal region side can be stably supported from the occipital region side by the loop-shaped (annular) contact surface into which the middle portion of the occipital region is fitted. In addition, the holder fixing portion can be mounted with the middle portion of the rounded occipital region fitted inside the loop shape, and thus the head is allowed to fit more in the holder fixing portion even in the event of individual head shape differences. As a result, the holder portion on the frontal side and the holder fixing portion on the occipital side are allowed to be in closer contact with the head of the subject, and thus the probe holder can be fixed to the head of the test subject in a more stable manner. Therefore, the probe holder can be fixed in a time-saving manner during brain function measurement and variations in brain function measurement result depending on fixing states can be suppressed.

With the present invention, a probe holder that can be fixed to a test subject's head in a more stable manner as described above and a brain function measurement device that is provided with the probe holder can be provided.

### BRIEF DESCRIPTION OF THE DRAWINGS

FIG. 1 is a block diagram illustrating a brain function measurement device and a probe holder according to an embodiment of the present invention;

FIG. 2 is a perspective view illustrating the probe holder according to the embodiment of the present invention from an occipital region side;

FIG. 3 is a perspective view illustrating the probe holder according to the embodiment of the present invention from a temporal region side;

FIG. 4 is a diagram illustrating the probe holder according to the embodiment of the present invention as a whole;

FIG. 5 is a schematic diagram for describing a positional relationship between a subject's occipital region and the probe holder;

FIG. 6(A) is a schematic sectional view illustrating a second adjustment portion in a state where a connecting unit is fixed;

FIG. 6(B) is a schematic sectional view illustrating the second adjustment portion in a state where the connecting unit is movable;

FIG. 7 is a schematic perspective view illustrating a first modification example of the probe holder according to the embodiment of the present invention; and

FIG. 8 is a schematic perspective view illustrating a second modification example of the probe holder according to the embodiment of the present invention.

### DETAILED DESCRIPTION OF THE DRAWINGS

Hereinafter, a specific embodiment of the present invention will be described with reference to accompanying drawings.

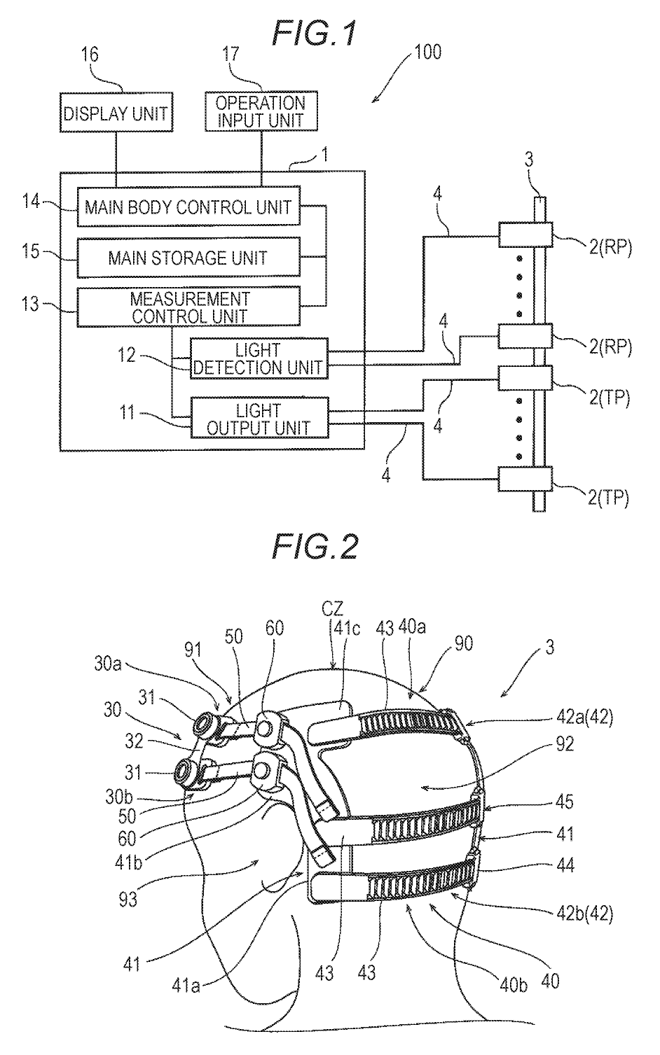

A brain function measurement device 100 and a probe holder 3 according to the embodiment of the present invention will be described with reference to FIGS. 1 to 6. In the present embodiment, an example in which the present invention is applied to an optical measurement device performing brain function measurement by irradiating a subject with measuring light as an example of brain function measurement devices will be described.

[Configuration of Brain Function Measurement Device]

An overall configuration of the brain function measurement device 100 will be described first with reference to FIG. 1.

The brain function measurement device 100 is provided with a main body portion 1, a plurality of measuring probes 2 (a light transmitting probe TP and a light receiving probe RP) connected to the main body portion 1, and the probe holder 3 for fixing the measuring probe 2 to a head 90 (refer to FIG. 2) of the subject. The main body portion 1 includes a light output unit 11 emitting the measuring light to the head 90 of the subject via the measuring probe 2 and a light detection unit 12 receiving the measuring light passing through the head 90 of the subject via the measuring probe 2. The light output unit 11 is provided with, for example, semiconductor laser as a light source and is connected to the light transmitting probe TP via an optical fiber cable 4 (hereinafter, referred to as an optical fiber 4). The light detection unit 12 is provided with, for example, a photomultiplier tube as a detector and is connected to the light receiving probe RP via the optical fiber 4.

In addition, the main body portion 1 includes a measurement control unit 13 performing operation control on the light output unit 11 and the light detection unit 12, a main body control unit 14 executing measurement operation control for the entire brain function measurement device 100 by executing various programs, and a main storage unit 15 storing the various programs executed by the main body control unit 14 and measurement data obtained as a result of measurement. In addition, the brain function measurement device 100 is provided with a display unit 16 and an operation input unit 17 connected to the main body portion 1. N+M is the total number of connectable probes of the brain function measurement device 100, and up to N light transmitting probes TP and up to M light receiving probes RP are connectable.

Although the plurality of measuring probes 2 is composed of the light transmitting probe TP and the light receiving probe RP in terms of function, the light transmitting probe TP and the light receiving probe RP are similar in terms of configuration (structure). The measuring probe 2 is a tubular terminal portion through which the optical fiber 4 passes, and the measuring probe 2 is configured such that a tip portion of the optical fiber 4 is exposed from a tip of the measuring probe 2. As a result, the measuring probe 2 emits the measuring light to the head of the subject from a tip portion or receives the measuring light emitted from the inside of the head.

The brain function measurement device 100 optically measures the subject's brain function by using near infrared spectroscopy (NIRS) and generates time-series measurement result data. Specifically, the brain function measurement device 100 emits the measuring light from the light transmitting probe TP attached to the probe holder 3 on the subject's head surface. Then, the intensity of the measuring light (a light receiving amount) is acquired by the measuring light reflected in the head being allowed to be incident on the light receiving probe RP attached to the probe holder 3 and being detected.

The brain function measurement device 100 measures the amounts of change in oxygenated hemoglobin, deoxygenated hemoglobin, and total hemoglobin based on the intensity of the measuring light (the light receiving amount) that has a plurality of wavelengths (for example, the three wavelengths of 780 nm, 805 nm, and 830 nm) and the light absorption characteristics of hemoglobin. As a result, the brain function measurement device 100 non-invasively measures a change in cerebral blood flow resulting from a brain activity as a change in hemoglobin amount.

In this manner, a brain activity measurement point (measurement channel) is formed between the light transmitting probe TP and the light receiving probe RP disposed on the head surface. The brain function measurement device 100 is capable of acquiring two-dimensional distribution on which brain region is active and how the brain region is active by measuring a wide brain region at a plurality of the measurement points (measurement channels) by using a plurality of the light transmitting probes TP and a plurality of the light receiving probes RP. Normally, each measuring probe 2 is attached to the probe holder 3 such that a matrix-shaped (matrix) arrangement in which the light transmitting probe TP and the light receiving probe RP are alternately arranged is formed so that the number of the measurement channels per measuring probe is increased.

[Configuration of Probe Holder]

A configuration of the probe holder 3 according to the present embodiment will be described below.

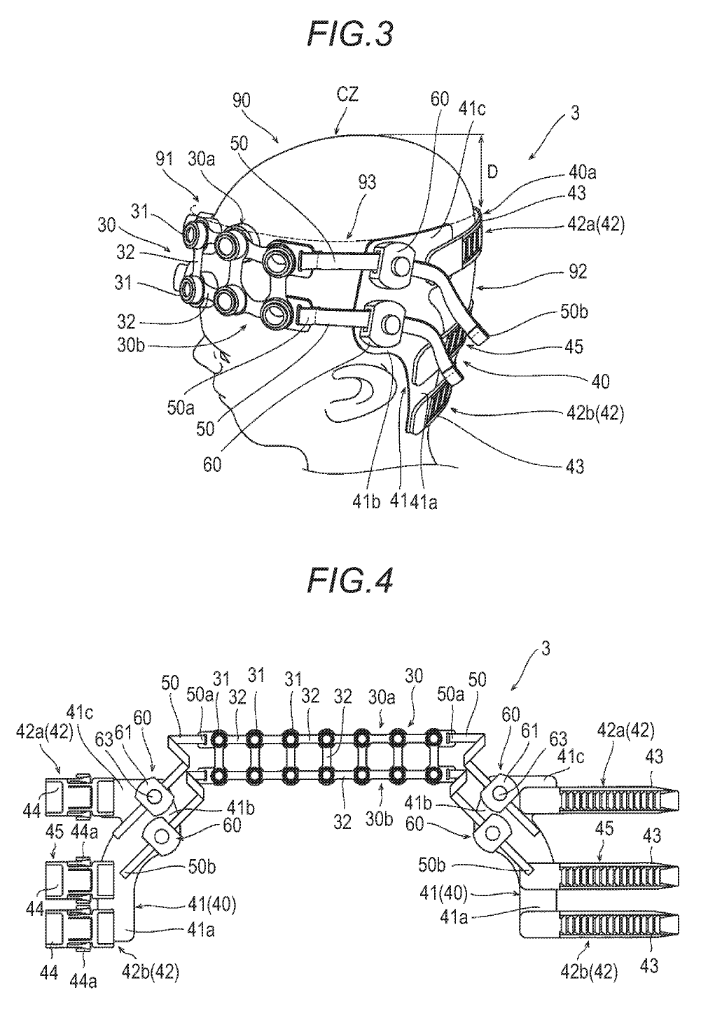

The probe holder 3 is a member for fixing the measuring probe 2 (refer to FIG. 1), and the probe holder 3 is configured to be mounted on the head 90 of the subject during the brain function measurement as illustrated in FIGS. 2 and 3. Each measuring probe 2 is held at a predetermined measurement position on the test subject's head surface by the probe holder 3. The description of the probe holder 3 will be based on the position of the probe holder 3 in a state where the probe holder 3 is mounted on the head 90 of the subject. Accordingly, the upward direction is the direction toward a centriciput region (CZ) and the left-right direction is the direction toward ear (temporal region) sides from the midline. The downward direction is the direction toward a cervical region side (body side).

As illustrated in FIGS. 2 to 4, the probe holder 3 is provided with a holder portion 30, a holder fixing portion 40, and a connecting unit 50. The holder portion 30 is disposed on the subject's frontal region 91 side. The holder fixing portion 40 is disposed on the subject's occipital region 92 side. The connecting unit 50 is disposed on the subject's left and right temporal region 93 sides and connects the holder portion 30 on the frontal region 91 side and the holder fixing portion 40 on the occipital region 92 side to each other. In this manner, the probe holder 3 is configured to make a circle around the subject's frontal region 91, temporal region 93, and occipital region 92 and is fixed to the head 90 by tightening (a tensile force) in a circumferential direction.

As illustrated in FIG. 4, the holder portion 30 includes a plurality of probe attachment portions 31 for attaching the measuring probe 2 of the brain function measurement device 100 and a connecting unit 32 connecting the probe attachment portions 31 to each other. The holder portion 30 is the part of the probe holder 3 to which the measuring probe 2 (refer to FIG. 1) is attached.

Each probe attachment portion 31 has a tubular structure in which a through-hole is formed and is configured to be capable of holding one inserted measuring probe 2. The plurality of probe attachment portions 31 is arranged in a matrix shape (matrix) at predetermined intervals. In the present embodiment, 14 probe attachment portions 31 in total are disposed in two rows and seven columns in the holder portion 30. Measurement channels of 19 points can be configured in a case where seven light transmitting probes TP and seven light receiving probes RP are alternately attached to the respective probe attachment portions 31. Still, the arrangement of the probe attachment portions 31 is not limited thereto. For example, the probe attachment portions 31 may also be arranged in any number of rows other than two and may also be arranged in any number of columns other than seven. Likewise, the probe attachment portions 31 may not be arranged in a matrix. Still, the matrix arrangement is preferable from the viewpoint of increasing the number of the measurement channels per measuring probe as described above.

The connecting unit 32 is a plate-shaped member connecting the probe attachment portions 31 to each other. The interval between the probe attachment portions 31 is maintained and the probe attachment portions 31 are interconnected by the connecting unit 32. The connecting unit 32 is formed of a resin material that allows bending deformation, and examples of the resin material include sheet-shaped (plate-shaped) polypropylene (PP). As a result, the holder portion 30 has a rectangular shape as a whole and is configured to be deformable in accordance with the subject's head shape. The connecting unit 32 is configured to undergo little stretching deformation and hold the probe attachment portions 31 at predetermined intervals.

In the present embodiment, the holder portion 30 is configured to be disposed on the forehead in the subject's frontal region 91. In a case where the number of rows of the probe attachment portions 31 increases, the holder portion 30 is disposed to become even closer to the centriciput CZ side from the forehead.

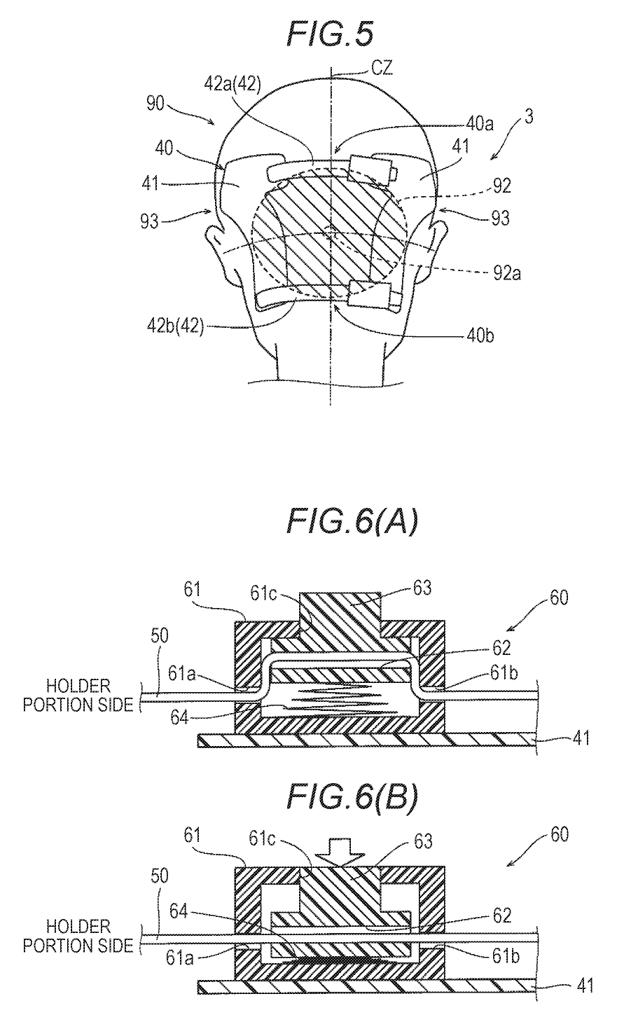

As illustrated in FIG. 2, the holder fixing portion 40 is disposed on the subject's occipital region 92 side and is formed in a loop shape (annular shape) such that a middle portion 92a (refer to FIG. 5) of the occipital region 92 is fitted into the holder fixing portion 40. The holder fixing portion 40 is the part of the probe holder 3 that supports the holder portion 30 from the subject's occipital region 92 side.

The holder fixing portion 40 includes a fixing member 41 connected to the holder portion 30 via the connecting unit 50 and a first adjustment portion 42 constituting a part of the loop shape by being connected to the fixing member 41 and configured to be adjustable in length.

A pair of the fixing members 41 is disposed as illustrated in FIG. 4 and is formed to extend in an up-down direction along both left and right sides of the occipital region 92 (refer to FIG. 5). The fixing member 41 is a sheet-shaped (plate-shaped) member that is formed of a resin material allowing bending deformation in accordance with the head shape, and examples of the resin material include sheet-shaped (plate-shaped) polypropylene (PP). The fixing member 41 is configured to undergo little stretching deformation within the range of the stress that acts when the fixing member 41 is used.

As illustrated in FIG. 3, the fixing member 41 integrally includes a lower side fixing portion 41a extending downward (to the cervical region side) along the back side of the subject's ear (hairline) during the mounting, an attachment portion 41b extending toward the temporal region 93 side from the subject's occipital region 92 side on the upper side of the ear, and an upper side fixing portion 41c extending backward (toward the rear midline) on the centriciput CZ side.

The lower side fixing portion 41a is formed to have a lower end position extending down to the lower portion of the occipital region 92. In other words, the lower side fixing portion 41a is formed to extend down to the inclined part between the middle portion 92a (refer to FIG. 5) of the occipital region 92 and the cervical region. The middle portion 92a of the occipital region 92 is the position of the occipital region that corresponds to the occipital protuberance. The attachment portion 41b is disposed between the upper side fixing portion 41c and the lower side fixing portion 41a. The attachment portion 41b is formed such that a tip on the frontal region 91 side protrudes up to a position (the temporal region 93) above the ear. The upper side fixing portion 41c is formed to protrude backward (toward the rear midline) from the upper end portion of the attachment portion 41b.

As illustrated in FIG. 2, the first adjustment portion 42 extends in the left-right direction in the occipital region 92 and connects the pair of fixing members 41 to each other by straddling the occipital region 92. As illustrated in FIG. 4, the first adjustment portion 42 is configured by a toothed strap 43 and a ratchet type buckle 44 engaged with the teeth of the toothed strap 43 being combined with each other. The toothed strap 43 is movable in an insertion direction and the toothed strap 43 is immovable in an extraction direction by the teeth meshing with the buckle 44 deviating every time the toothed strap 43 is sent in the insertion direction. By a release lever 44a of the buckle 44 being operated, the engagement between the toothed strap 43 and the buckle 44 is released and the toothed strap 43 can be moved in the extraction direction. In this manner, the first adjustment portion 42 is configured such that the length between the pair of fixing members 41 is adjustable.

A plurality of the first adjustment portions 42 is disposed, and the first adjustment portions 42 will be distinguished into first adjustment portions 42a and 42b in the following description. As illustrated in FIG. 2, the upper side first adjustment portion 42a is disposed to connect the upper side fixing portions 41c of the fixing members 41 to each other. The lower side first adjustment portion 42b is disposed to connect the lower end portions of the lower side fixing portions 41a of the pair of fixing members 41 to each other. In other words, the upper and lower end portions of the pair of fixing members 41 are connected to each other by the first adjustment portions 42a and 42b, respectively. The upper side first adjustment portion 42a and the lower side first adjustment portion 42b are disposed such that the middle portion 92a (refer to FIG. 5) of the occipital region 92 is positioned between the first adjustment portion 42a and the first adjustment portion 42b in the up-down direction.

In this manner, in the present embodiment, the holder fixing portion 40 is formed in a loop shape (annular shape), such that the middle portion 92a of the occipital region 92 is fitted into the holder fixing portion 40, by the pair of fixing members 41 and a pair of the first adjustment portions 42 (the upper side first adjustment portion 42a and the lower side first adjustment portion 42b). The length of the loop-shaped part of the holder fixing portion 40 can be adjusted by the lengths of the first adjustment portion 42a and the first adjustment portion 42b being adjusted.

In the present embodiment, an intermediate adjustment portion 45 is disposed between the first adjustment portions 42a and 42b. The intermediate adjustment portion 45 is disposed to connect the intermediate positions of the lower side fixing portions 41a of the fixing members 41 to each other. The intermediate adjustment portion 45 does not constitute a part of the loop shape and does not contribute to the adjustment of the length of the loop-shaped part. The intermediate adjustment portion 45 is further disposed to enhance the adhesiveness of the holder fixing portion 40 with respect to the head 90. The intermediate adjustment portion 45 is similar in structure to the first adjustment portion 42.

The mounting position of the holder fixing portion 40 will be described in further detail below. In the present embodiment, the holder fixing portion 40 is formed such that an upper portion 40a of the loop shape is disposed at substantially the same height as an upper end portion 30a of the holder portion 30 or closer to the centriciput CZ side than the upper end portion 30a of the holder portion 30 and a lower portion 40b of the loop shape is disposed on the lower portion side of the occipital region 92 on the head 90 of the subject.

In other words, as illustrated in FIG. 3, the first adjustment portion 42a and the upper side fixing portion 41c of the fixing member 41 constituting the upper portion 40a of the holder fixing portion 40 are positioned at substantially the same height as the upper end portion 30a of the holder portion 30 or closer to the centriciput CZ side than the upper end portion 30a of the holder portion 30 on the head 90 of the subject. In other words, a distance D from the centriciput CZ to the upper side fixing portion 41c (or the first adjustment portion 42a) is almost equal to a distance D from the centriciput CZ to the upper end portion 30a of the holder portion 30 or the upper side fixing portion 41c (or the first adjustment portion 42a) is closer to the centriciput CZ than the upper end portion 30a of the holder portion 30.

In addition, as illustrated in FIG. 5, the tip portion of the lower side fixing portion 41a of the fixing member 41 and the first adjustment portion 42b constituting the lower portion 40b of the holder fixing portion 40 are positioned in the lower portion of the occipital region 92 (inclined part between the middle portion 92a and the cervical region) on the head 90 of the subject.

Furthermore, in the present embodiment, the holder fixing portion 40 is formed in a loop shape surrounding the subject's occipital region 92 (indicated by hatching in FIG. 5). In other words, the holder fixing portion 40 surrounds the subject's occipital region 92 by the pair of fixing members 41 and the first adjustment portions 42a and 42b being disposed along both left and right side portions and the lower and upper portions of the occipital region 92, respectively. FIG. 5 is a diagram schematically illustrating the probe holder 3 with the connecting unit 50 and the intermediate adjustment portion 45 omitted.

The connecting unit 50 illustrated in FIG. 4 is a belt-shaped member in which an end 50a is fixed to the holder portion 30 and the other end 50b side is connected to the holder fixing portion 40. In the present embodiment, a second adjustment portion 60 for adjusting the connection length of the connecting unit 50 between the holder fixing portion 40 and the holder portion 30 is disposed in the holder fixing portion 40 and the other end 50b side of the connecting unit 50 is connected to the holder fixing portion 40 via the second adjustment portion 60.

The connecting unit 50 is disposed on each of the subject's left and right temporal region 93 sides, and a pair of the connecting units 50 is disposed on each temporal region 93 side such that each of the upper end portion 30a and a lower end portion 30b of the holder portion 30 is connected to the holder fixing portion 40. Accordingly, four connecting units 50 in total are disposed such that each of the four corners of the rectangular holder portion 30 is connected to the holder fixing portion 40.

The connecting unit 50 connects the attachment portion 41b of the fixing member 41 of the holder fixing portion 40 and the holder portion 30 to each other. In other words, the second adjustment portion 60 is disposed in the attachment portion 41b with the other end 50b side of the connecting unit 50 connected thereto. When the probe holder 3 is mounted, the length of each connecting unit 50 is adjusted such that a tightening force (tensile force) not causing the holder portion 30 and the holder fixing portion 40 disposed at appropriate positions to deviate acts between the holder portion 30 and the holder fixing portion 40.

The number of the second adjustment portions 60 is equal to the number of the connecting units 50. The second adjustment portion 60 is fixedly disposed in the attachment portion 41b of the fixing member 41.

As illustrated in FIGS. 6(A) and 6(B), the second adjustment portion 60 includes a case portion 61, a passage portion 62 through which the other end 50b of the connecting unit 50 passes, and a switching operation unit 63.

The case portion 61 is a box-shaped member in which an entrance opening 61a, an exit opening 61b, and an upper surface opening 61c are formed. The entrance opening 61a and the exit opening 61b are formed in a pair of side surfaces of the case portion 61 facing each other, respectively. Both the entrance opening 61a and the exit opening 61b are formed to have a predetermined size such that the connecting unit 50 passes.

The passage portion 62 is formed integrally with the switching operation unit 63 and is accommodated in the case portion 61. The passage portion 62 is a through-hole formed along the direction in which the entrance opening 61a and the exit opening 61b are connected to each other. The passage portion 62 is formed to have a predetermined size such that the connecting unit 50 passes. The connecting unit 50 passes the entrance opening 61a, the passage portion 62, and the exit opening 61b in sequence and the other end 50b goes outside (refer to FIG. 3) from the exit opening 61b.

The switching operation unit 63 has a function to switch between a state where the connecting unit 50 is fixed in the passage portion 62 (refer to FIG. 6(A)) and a state where the connecting unit 50 is movable in the passage portion 62 (refer to FIG. 6(B)). The switching operation unit 63 is a button (projection portion) disposed on the upper surface side of the passage portion 62 and is exposed to the outside from the inside of the upper surface opening 61c of the case portion 61. The switching operation unit 63 is biased from the inside of the case portion 61 toward the upper surface side of the case portion 61 along with the passage portion 62 by a biasing member 64.

As illustrated in FIG. 6(A), in a state where the switching operation unit 63 is not operated, the passage portion 62 is disposed at a position deviating from a straight line connecting the entrance opening 61a and the exit opening 61b of the case portion 61 to each other by the biasing member 64 and the connecting unit 50 therein is bent and fixed. Once the biasing member 64 is compressed by the switching operation unit 63 being pushed as illustrated in FIG. 6(B), the passage portion 62 is disposed on the straight line connecting the entrance opening 61a and the exit opening 61b of the case portion 61 to each other and the connecting unit 50 therein becomes movable in the passage portion 62.

The following effects can be achieved with the present embodiment.

In the present embodiment, a loop-shaped contact surface can be formed on the test subject's occipital region 92 side by the holder fixing portion 40 since the holder fixing portion 40 disposed on the subject's occipital region 92 side and formed in a loop shape such that the middle portion 92a of the occipital region 92 is fitted into the holder fixing portion 40 is disposed as described above. As a result, the holder portion 30 on the frontal region 91 side can be stably supported from the occipital region 92 side by the loop-shaped (annular) contact surface into which the middle portion 92a of the occipital region 92 is fitted unlike in a case where a belt-shaped contact surface simply for placing a belt like a bridge is formed in the occipital region 92. In addition, the holder fixing portion 40 can be mounted with the middle portion 92a of the rounded occipital region 92 fitted inside the loop shape, and thus the head 90 is allowed to fit more in the holder fixing portion 40 than in conventional structures even in the event of individual head shape differences. As a result, the holder portion 30 and the holder fixing portion 40 are allowed to be in closer contact with the head 90 of the subject, and thus the probe holder 3 can be fixed to the head 90 of the test subject in a more stable manner.

In addition, in the present embodiment, the holder fixing portion 40 is formed such that the upper portion 40a of the loop shape is disposed at the same height as the upper end portion 30a of the holder portion 30 or closer to the centriciput CZ side than the upper end portion 30a of the holder portion 30 and the lower portion 40b of the loop shape is disposed on the lower portion side of the occipital region 92 on the head 90 of the subject as described above. Accordingly, the holder fixing portion 40 can be disposed in a wider range of the head 90 of the test subject in the up-down direction, and thus the holder fixing portion 40 can be fixed to the head 90 of the test subject in a more stable manner. As a result, deviation of the probe holder 3 from the head 90 during the mounting can be suppressed.

In addition, in the present embodiment, the holder fixing portion 40 is formed in a loop shape surrounding the subject's occipital region 92 as described above. As a result, the holder fixing portion 40 can be mounted with the entire occipital region 92 fitted inside the loop shape, and thus the stability of the holder fixing portion 40 during the mounting can be further improved.

In addition, in the present embodiment, the fixing member 41 and the first adjustment portion 42 constituting a part of the loop shape and adjustable in length are disposed in the holder fixing portion 40 as described above. As a result, the length of the loop-shaped part of the holder fixing portion 40 can be adjusted in accordance with the subject's head shape, and thus the probe holder 3 can be fixed to the head 90 of the test subject in a more stable manner even in the event of individual head shape differences.

In addition, in the present embodiment, the connecting unit 50 is disposed on each of the subject's left and right temporal region 93 sides and the pair of connecting units 50 is disposed on each temporal region 93 side such that each of the upper end portion 30a and the lower end portion 30b of the holder portion 30 is connected to the holder fixing portion 40 as described above. Accordingly, each of the upper end portion 30a and the lower end portion 30b of the holder portion 30 can be supported by the holder fixing portion 40 on the occipital region 92 side via the connecting unit 50. As a result, the subject's frontal region 91 is allowed to fit more in the holder portion 30 when the holder portion 30 is fixed, and thus the occurrence of positional deviation of the holder portion 30 (measuring probe 2) during the mounting of the probe holder 3 can be effectively suppressed.

In addition, in the present embodiment, the attachment portion 41b extending from the subject's occipital region 92 side toward the temporal region 93 side is disposed in the holder fixing portion 40 as described above. The connecting unit 50 is disposed to connect the attachment portion 41b and the holder portion 30 to each other. Accordingly, the attachment portion 41b extending from the occipital region 92 side to the temporal region 93 side can be connected to the holder portion 30 on the frontal side, and thus the length of the connection between the holder portion 30 and the holder fixing portion 40 by the connecting unit 50 can be decreased. A tensile force (that is, a force to tighten the head 90 of the test subject) acts on the connecting unit 50 so that the holder portion 30 and the holder fixing portion 40 are fixed to the head 90, and thus a large tensile force is allowed to act with ease as a result of the decrease in connection length. As a result, the holder portion 30 and the holder fixing portion 40 can be fixed in close contact to the head 90 in a more appropriate manner.

In addition, in the present embodiment, the second adjustment portion 60 for adjusting the connection length of the connecting unit 50 between the holder fixing portion 40 and the holder portion 30 is disposed and the passage portion 62 through which the other end 50b of the connecting unit 50 passes and the switching operation unit 63 switching between the state where the connecting unit 50 is fixed in the passage portion 62 and the state where the connecting unit 50 is movable in the passage portion 62 are disposed in the second adjustment portion 60 as described above. Accordingly, during the mounting of the probe holder 3, the connection length of the connecting unit 50 can be easily and conveniently adjusted simply by the switching operation unit 63 being operated. As a result, the probe holder 3 can be fixed to the head 90 in a more stable manner, by the holder portion 30 and the holder fixing portion 40 connected at an appropriate connection length, even in the case of subjects' individual head shape differences.

It should be noted that the embodiment disclosed above is illustrative in all respects and is not restrictive. The scope of the present invention is indicated not by the above description of the embodiment but by the scope of claims and also includes every modification within the meaning and range equivalent to the scope of claims.

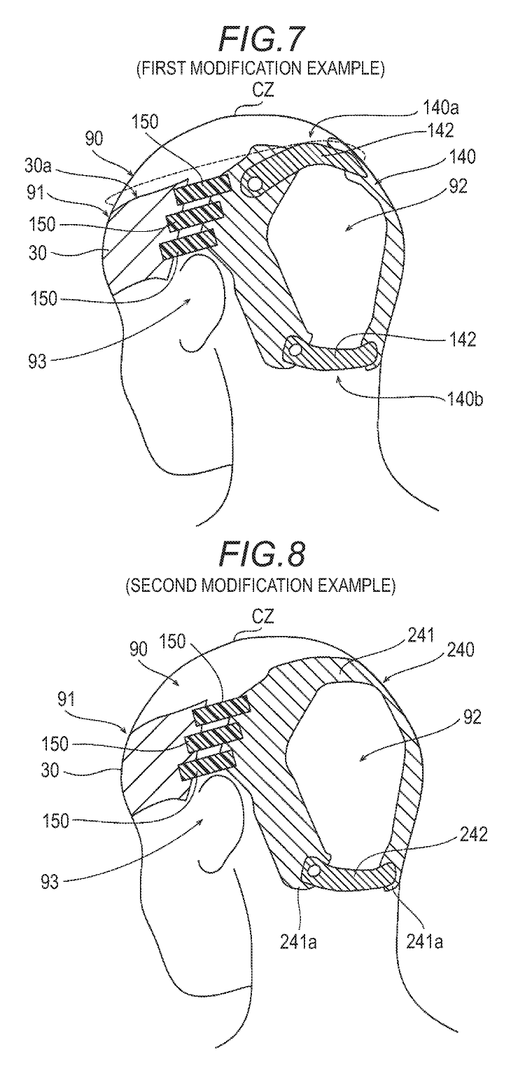

For example, although an example in which the upper portion 40a of the holder fixing portion 40 is configured to be at substantially the same height as the upper end portion 30a of the holder portion 30 has been described in the embodiment above, the present invention is not limited thereto. In the present invention, an upper portion 140a of a holder fixing portion 140 may also be disposed at a position closer to the centriciput CZ than the upper end portion 30a of the holder portion 30 as in a first modification example illustrated in FIG. 7. In FIGS. 7 and 8, each portion of a probe holder is indicated by hatching for convenience.

In addition, although an example in which each of the upper and lower end portions of the pair of fixing members 41 of the holder fixing portion 40 is connected by the first adjustment portion 42 has been described in the embodiment above, the present invention is not limited thereto. In the present invention, a single inverted U-shaped fixing member 241 surrounding the occipital region 92 may also be disposed and a pair of lower end portions 241a of the fixing member 241 may also be connected to each other by a first adjustment portion 242 as in a holder fixing portion 240 according to a second modification example illustrated in FIG. 8. A pair of upper end portions of a single U-shaped fixing member may be connected to each other by a first adjustment portion as well.

In addition, although an example in which two, upper and lower, first adjustment portions 42 are disposed has been described in the embodiment above, the present invention is not limited thereto. In the present invention, the single first adjustment portion 242 may also be disposed as in the second modification example illustrated in FIG. 8. In a case where the upper and lower first adjustment portions are disposed as in the embodiment above and the first modification example (refer to FIG. 7), the shape of the loop can be adjusted more freely and the length of the loop-shaped part can still be adjusted, and thus head shape variations (individual head shape differences) can be responded to in a more appropriate manner. Even in a case where the upper side of the occipital region 92 has a large width and even in a case where the upper side of the occipital region 92 has a small width, for example, the head is allowed to fit more by the length of the first adjustment portion being adjusted. In addition, the first adjustment portion is optional in the present invention.

In addition, although an example in which the first adjustment portion 42 of the holder fixing portion 40 is configured by the toothed strap 43 and the ratchet type buckle 44 being combined with each other has been described in the embodiment above, the present invention is not limited thereto. In the present invention, a hook and loop fastener or the like may also be used in the first adjustment portion. In the first modification example illustrated in FIG. 7 and the second modification example illustrated in FIG. 8, examples are shown in which a first adjustment portion 142 (the first adjustment portion 242) is composed of a hook and loop fastener. Alternatively, length adjustment may be performed with a string-shaped first adjustment portion connected or a first adjustment portion may be configured by a buckle and a belt having no ratchet structure being combined with each other. In addition, a first adjustment portion may be composed of the same adjustment mechanisms as in the second adjustment portion 60 (the passage portion 62 and the switching operation unit 63) and a belt-shaped member.

Likewise, although an example in which length adjustment can be performed by the connecting unit 50 as a belt-shaped member being connected to the second adjustment portion 60 has been described in the embodiment above, the present invention is not limited thereto. In the present invention, a hook and loop fastener or the like may also be used in the connecting unit. In the first modification example illustrated in FIG. 7 and the second modification example illustrated in FIG. 8, examples are shown in which the unit connector 150 is composed of a hook and loop fastener. In addition, length adjustment may be performed with a string-shaped connecting unit connected or the length of the connecting unit may be adjusted based on a combination between a belt (connecting unit) and a buckle (second adjustment portion).

In addition, although an example in which the connecting unit 50 is disposed such that each of the upper end portion 30a and the lower end portion 30b of the holder portion 30 is connected to the holder fixing portion 40 has been described in the embodiment above, the present invention is not limited thereto. In the present invention, three or more connecting units 150 may also be disposed such that the intermediate part of the holder portion 30 as well as the upper and lower end portions is connected to the holder fixing portion 140 (240) as in, for example, the first modification example illustrated in FIG. 7 and the second modification example illustrated in FIG. 8.

In addition, although an example in which the holder fixing portion 40 is formed in a loop shape surrounding the subject's occipital region 92 has been described in the embodiment above, the present invention is not limited thereto. In the present invention, the holder fixing portion may have any loop shape into which at least the middle portion 92a (the occipital protuberance) of the subject's occipital region 92 is fitted. In other words, the holder fixing portion may have any loop shape surrounding the middle portion 92a (refer to FIG. 5) of the occipital region 92 and the loop shape may also be a shape surrounding a predetermined range around the middle portion 92a. Still, in a case where the holder fixing portion is formed such that substantially the entire occipital region is surrounded by the loop shape being enlarged, a wider contact area can be ensured and the probe holder can be fixed in a more stable manner as a result.

In addition, although an example in which the attachment portion 41b extending from the subject's occipital region 92 side toward the temporal region 93 side is disposed in the holder fixing portion 40 and the connecting unit 50 is configured to connect the attachment portion 41b and the holder portion 30 to each other has been described in the embodiment above, the present invention is not limited thereto. In the present invention, the holder portion and the holder fixing portion may also be connected to each other by the connecting unit extending up to the occipital region side without the attachment portion extending up to the temporal region side.

1. A probe holder for a brain function measurement device, the probe holder comprising:
a holder portion including a probe attachment portion for attaching a measuring probe of the brain function measurement device, disposed on a subject's frontal region side, and deformable in accordance with the subject's head shape;
a holder fixing portion disposed on the subject's occipital region side and formed in a loop shape such that a middle portion of the occipital region is fitted into the holder fixing portion; and
a connecting unit connecting the holder portion on the frontal region side and the holder fixing portion on the occipital region side to each other.

2. The probe holder for a brain function measurement device according to claim 1,
wherein the holder fixing portion is formed such that an upper portion of the loop shape is disposed at the same height as an upper end portion of the holder portion or closer to a centriciput side than the upper end portion of the holder portion and a lower portion of the loop shape is disposed on a lower portion side of the occipital region on the subject's head.

3. The probe holder for a brain function measurement device according to claim 1,
wherein the holder fixing portion is formed in the loop shape surrounding the subject's occipital region.

4. The probe holder for a brain function measurement device according to claim 1,
wherein the holder fixing portion includes a fixing member connected to the holder portion via the connecting unit and a first adjustment portion constituting a part of the loop shape by being connected to the fixing member and configured to be adjustable in length.

5. The probe holder for a brain function measurement device according to claim 1,
wherein the connecting unit is disposed on each of the subject's left and right temporal region sides and a pair of the connecting units is disposed on each temporal region side such that each of the upper end portion and a lower end portion of the holder portion is connected to the holder fixing portion.

6. The probe holder for a brain function measurement device according to claim 1,
wherein the holder fixing portion includes an attachment portion extending from the subject's occipital region side toward the temporal region side, and
wherein the connecting unit connects the attachment portion and the holder portion to each other.

7. The probe holder for a brain function measurement device according to claim 1,
wherein the connecting unit is a belt-shaped member in which one end is fixed to one of the holder fixing portion and the holder portion and the other end side is connected to the other one of the holder fixing portion and the holder portion,
wherein a second adjustment portion disposed in the other one of the holder fixing portion and the holder portion for adjusting a connection length of the connecting unit between the holder fixing portion and the holder portion is further provided, and
wherein the second adjustment portion includes a passage portion through which the other end of the connecting unit passes and a switching operation unit switching between a state where the connecting unit is fixed in the passage portion and a state where the connecting unit is movable in the passage portion.

8. A brain function measurement device comprising:
a light output unit irradiating a subject's head with measuring light via a measuring probe;
a light detection unit receiving the measuring light passing through the subject's head via the measuring probe; and
a probe holder for fixing the measuring probe to the subject's head,
wherein the probe holder includes
a holder portion including a probe attachment portion for attaching the measuring probe, disposed on the subject's frontal region side, and deformable in accordance with the subject's head shape,
a holder fixing portion disposed on the subject's occipital region side and formed in a loop shape such that a middle portion of the occipital region is fitted into the holder fixing portion, and
a connecting unit connecting the holder portion on the frontal region side and the holder fixing portion on the occipital region side to each other.
